(19) United States
(12) Patent Application Publication
(10) Pub. No.: US 2026/0098004 A1
(43) Pub. Date: Apr. 9, 2026

(54) PLASTIC UPCYCLING SYSTEM DESIGNATED FOR MARINE PLASTIC WASTE

(71) Applicants: The Johns Hopkins University, Baltimore, MD (US); Stamato Solutions LLC, Califon, NJ (US)

(72) Inventors: Chao WANG, Baltimore, MD (US); Han ZONG, Baltimore, MD (US); Noah ZECHER-FREEMAN, Baltimore, MD (US); Howard STAMATO, Baltimore, MD (US)

(21) Appl. No.: 19/416,294

(22) Filed: Dec. 11, 2025

Related U.S. Application Data
(63) Continuation of application No. PCT/US2024/035215, filed on Jun. 24, 2024.
(60) Provisional application No. 63/510,004, filed on Jun. 23, 2023.

(51) Int. Cl. C07C 4/06; B01J 19/00; B01J 19/24

(52) U.S. Cl. CPC C07C 4/06; B01J 19/0013; B01J 19/2415; B01J 2219/00157

(57) ABSTRACT

An upcycling system for converting plastic waste to value-added aromatic products and process of using same. The system includes a plug-flow reactor with catalytic hydrocracking functionality. Zeolite catalysts can be used to convert the plastic waste, for example marine plastic waste, into aromatic products such as benzene, toluene and/or xylene.

### CROSS-REFERENCE TO RELATED APPLICATIONS

This application is a continuation application claiming priority to International Patent Application No. PCT/US2024/035215 filed on 24 Jun. 2024, which claims priority to U.S. Provisional Application No. 63/510,004, filed Jun. 23, 2023, the contents of which are incorporated herein by reference in its entirety.

### BACKGROUND

Polymers are irreplaceable in the global economy, with a myriad of uses in packaging, construction, transportation, electronics, and health-care industries. Many of these applications rely on plastics as inexpensive disposable materials, which are nonetheless often precisely constructed to confer desired properties essential for the targeted function. Their massive-scale manufacture, single-use function, long lifetimes, slow decomposition rates, and disruption of sensitive ecosystems, however, have created a crisis of plastics waste.

In light of both the large drain on global resources and the massive amount of waste material generated, plastics represent a tremendous and as-yet-untapped domestic resource for the production of chemicals and new materials. Efficient technologies for extracting this value from discarded polymers would be equivalent to recovering about 3.5 billion barrels of oil each year and could create entirely new industries. Currently, most of the stored energy in plastics is irreversibly lost into landfills that are overflowing throughout our planet. While physical recycling is desirable and widespread in many areas for a wide range of materials, it is most effective for recovering glass, paper, and metals such as aluminum. Recycling, to date, has not been able to efficiently and cleanly recoup the inherent value in plastics, especially low-density polyethylene (plastic bags), polypropylene, and polystyrene. The syntheses of many virgin plastics are currently less expensive than creating quality recycled materials, because plastics are made on a large scale in centralized plants, from inexpensive monomers such as ethylene, in processes that are tightly integrated into commodity chemical production. As a result, many plastics are just burned as fuel or inefficiently reprocessed to manufacture lower-value materials (known as downcycling), resulting in minimal economic incentives for waste recovery, sorting, and processing.

Unfortunately, many plastics are not recycled and end up in the oceans. Some reports state that between 75 and 199 million tons of plastic is polluting our oceans. Rather than evenly dispersing in the oceans, plastics tend to concentrate in one of five gyres. Current estimates of the total size of the northern pacific gyre, the largest gyre, is that it covers more than 15,000,000 square kilometers. In addition, these plastics are broken down into small pieces by ultraviolet light and fluid power. These plastics and microplastics, which are tens of microns to several millimeters in size, are accidentally introduced into the body of fish and other aquatic organisms as food. It has been reported that some marine organisms die as a result. In addition, microplastics may have harmful substances attached to them, and there are concerns that eating fish that contain such microplastics may have an enormous impact on human health.

Chemical upcycling, an emerging alternative to the classical recycling approach, would use plastic waste as a feedstock for the synthesis of value-added chemicals and materials. Disadvantageously, most plastic collected today is not deemed suitable for upcycling due, in part, to the variability of the incoming plastic stream. The sources of the variability include the mixing of different types of plastics, the degree of degradation associated with reprocessing plastics, and the presence of low molecular weight compounds.

There continues to be a need for a system and process that can upcycle plastic waste including, but not limited to, marine plastic waste, wherein the plastic waste is converted to value-added aromatic products such as benzene, toluene, and xylene (BTX).

### SUMMARY

In one aspect, a system for upcycling plastic waste to produce aromatic compounds is described, said system comprising:
a plug-flow reactor (PFR) with catalytic hydrocracking functionality, wherein the PFR comprises a reactor tube comprising a catalytic material;
a furnace;
a source of inert gas;
a source of hydrogen gas; and
a cold trap.

In another aspect, a process of upcycling plastic waste to produce aromatic compounds is described, said process comprising:
introducing the plastic waste to plug-flow reactor (PFR) reactor tube, wherein the reactor tube comprises a catalytic material;
removing air from the reactor tube using at least one inert gas;
heating the reactor tube and the plastic waste contained therein to a target temperature;
introducing a feeding gas to the reactor tube once the target temperature is achieved; and
collecting the produced aromatic compounds through solvent extraction in a cold trap.

Other aspects, features and advantages of the invention will be more fully apparent from the ensuing disclosure and appended claims.

### BRIEF DESCRIPTION OF THE FIGURES

FIG. 1. A schematic of the upcycling system described herein.

FIG. 2. Upcycling performance results obtained using the system described herein.

### DETAILED DESCRIPTION OF THE DISCLOSURE

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,”the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, a “system” refers to a plurality of real and/or abstract elements operating together for a common purpose. In some embodiments, a “system” is an integrated assemblage of hardware and/or software elements. In some embodiments, each component of the system interacts with one or more other elements and/or is related to one or more other elements. In some embodiments, a system refers to a combination of components and software for controlling and directing methods.

As used herein, the terms “aromatics” or “aromatic compound” are used to refer to a hydrocarbon compound or compounds comprising one or more aromatic groups such as, for example, single aromatic ring systems (e.g., benzyl, phenyl, etc.) and fused polycyclic aromatic ring systems (e.g. naphthyl, 1,2,3,4-tetrahydronaphthyl, etc.). Examples of aromatic compounds include, but are not limited to, benzene, toluene, indane, indene, 2-ethyl toluene, 3-ethyl toluene, 4-ethyl toluene, trimethyl benzene (e.g., 1,3,5-trimethyl benzene, 1,2,4-trimethyl benzene, 1,2,3-trimethyl benzene, etc.), ethylbenzene, styrene, cumene, methylbenzene, propylbenzene, xylenes (e.g., p-xylene, m-xylene, o-xylene, etc.), naphthalene, methyl-naphthalene (e.g., 1-methyl naphthalene, anthracene, 9.10-dimethylanthracene, pyrene, phenanthrene, dimethyl-naphthalene (e.g., 1,5-dimethylnaphthalene, 1,6-dimethylnaphthalene, 2,5-dimethylnaphthalene, etc.), ethyl-naphthalene, hydrindene, methyl-hydrindene, and dymethyl-hydrindene. Single-ring and/or higher ring aromatics may also be produced in some embodiments.

The terms “plastics” and “polymers” are used interchangeably herein. A polymer is a carbon-based (at least 50 mass % C) material chiefly made up of repeating units and having a number average molecular weight of at least 100, typically greater than 1000 or greater than 10,000.

The term “fluidized bed reactor” is given its conventional meaning in the art and is used to refer to reactors comprising a vessel that can contain a granular solid material (e.g., silica particles, catalyst particles, etc.), in which a fluid (e.g., a gas or a liquid) is passed through the granular solid material at velocities sufficiently high as to suspend the solid material and cause it to behave as though it were a fluid.

The term “plug flow reactor,” also known as tubular reactors or continuous tubular reactors, is used to refer to reactors wherein reactants continuously flow through a tube or pipe as if they were a “plug” of fluid, with no back-mixing between different portions of the fluid, and products exit the reactor. The reactor contents are not continuously stirred but instead as the plug of fluid flows through the tube or pipe, reactants are converted to products. In some embodiments, the interior surface of the reactor comprises catalytically active surfaces. The chemical reaction occurs within the tube or pipe of the plug-flow reactor. In some embodiments, the heat within the reactor tube is substantially isothermal. In some embodiments, the reactor tube is immersed in a bath or a heat transfer medium (e.g., molten salt), or positioned within a jacket so as to maintain a substantially isothermal reaction within the reactor tube. In some embodiments, the contents of the reactor tube are heated using any means known in the art. In some embodiments, the contents of the reactor tube are heated using a furnace. In some embodiments, the reactor tube is substantially straight. In some embodiments, the reactor tube is substantially coiled.

The terms “olefin” or “olefin compound” (a.k.a. “alkenes”) are given their ordinary meaning in the art and are used to refer to any unsaturated hydrocarbon containing one or more pairs of carbon atoms linked by a double bond. Olefins include both cyclic and acyclic (aliphatic) olefins, in which the double bond is located between carbon atoms forming part of a cyclic (closed-ring) or of an open-chain grouping, respectively. In addition, olefins may include any suitable number of double bonds (e.g., monoolefins, diolefins, triolefins, etc.). Examples of olefin compounds include, but are not limited to, ethene, propene, allene (propadiene), 1-butene, 2-butene, isobutene (2 methyl propene), butadiene, and isoprene, among others. Examples of cyclic olefins include cyclopentene, cyclohexane, cycloheptene, among others. Aromatic compounds such as toluene are not considered olefins; however, olefins that include aromatic moieties are considered olefins, for example, benzyl acrylate or styrene.

As used herein, an “inert gas” includes, but is not limited to, nitrogen, helium, argon, neon, xenon, krypton, and radon.

The term “yield” is used herein to refer to the amount of a product flowing out of a reactor divided by the amount of reactant flowing into the reactor, usually expressed as a percentage or fraction. Yields are often calculated on a mass basis or on the basis of a particular feed component. Mass yield is the mass of a particular product divided by the weight of feed used to prepare that product. For example, if 500 grams of polymer is fed to a reactor and 45 grams of benzene is produced, the mass yield of benzene would be 45/500=9% benzene.

The term “plastic waste,” as used herein, includes at least one of the major thermoplastic resins such as polyethylene (low density, linear low density, and high density), polypropylene, polyethylene terephthalate, polystyrene, and polyvinyl chloride. It should be appreciated that the plastic waste can include terrestrial plastic waste, for example as collected by recycling facilities, or marine plastic waste, for example, floating marine plastic waste or plastic waste collected from the bottom of a body of water. Methods of collection of marine plastic waste are known in the art.

Broadly, the present invention provides a robust upcycling system designated for plastic waste including, but not limited to, marine plastic waste. This system comprises a plug-flow reactor (PFR) with catalytic hydrocracking functionality. The system further comprises zeolite-base catalysts to convert plastic waste into value-added aromatic products including, but not limited to, benzene, toluene and xylene (BTX). In some embodiments, the upcycling system is portable.

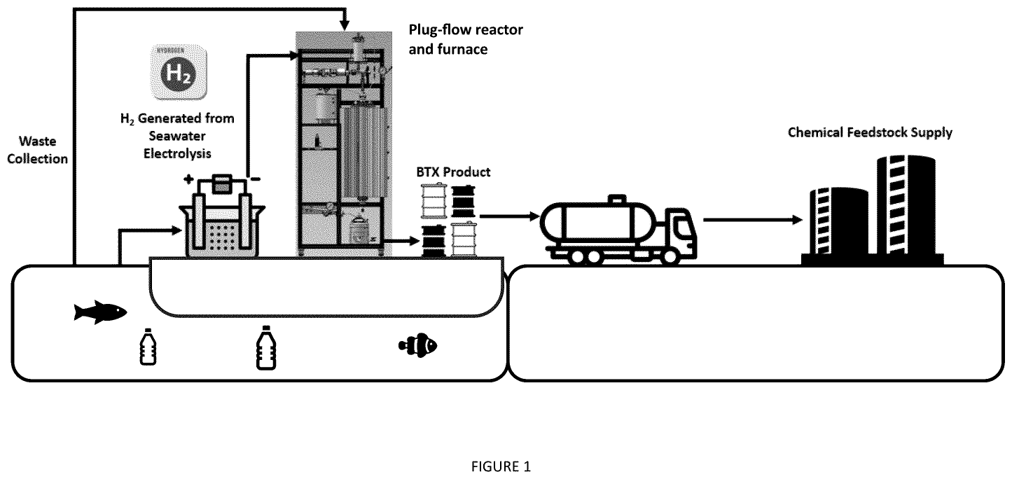

Referring to FIG. 1, an embodiment of the system is illustrated. Plastic waste, e.g., marine plastic waste, is introduced to a plug-flow reactor maintained substantially at atmospheric pressure. The PFR comprises a reactor tube, wherein the reactor tube comprises a catalyst material capable of hydrocracking the components of the plastic waste into value-added aromatic products, wherein the catalyst material coats or lines an interior surface of the reactor tube. In some embodiments, the catalyst material comprises a zeolite. In some embodiments, the plastic waste is introduced to the reactor tube comprising the catalyst. A furnace, e.g., a tubular furnace, or some other heating means is used to heat the contents of the reactor tube. In some embodiments, the furnace or other heating means accommodates the reactor tube within an interior of the furnace. In some embodiments, the furnace or other heating means is separate from the reactor tube and the heat generated from the furnace or other heating means is used to heat the reactor tube and the contents of the reactor tube, as understood by the person skilled in the art.

Inert gas is introduced to the reactor tube to remove any remaining air and maintain an inert gas atmosphere therein. Once an inert gas atmosphere is achieved in the reactor tube, the reactor tube is heated to a target reaction temperature. In some embodiments, the target reaction temperature is in a range from about 500° C. to about 900° C., or about 500° C. to about 550° C., or about 550° C. to about 600° C., or about 600° C. to about 650° C., or about 650° C. to about 700° C., or about 700° C. to about 750° C., or about 750° C. to about 800° C., or about 800° C. to about 850° C., or about 850° C. to about 900° C., or about 500° C. to about 600° C., or about 600° C. to about 700° C., or about 700° C. to about 800° C., or about 800° C. to about 900° C. As soon as the target reaction temperature is achieved, a feeding gas can be introduced to the reactor tube. In some embodiments, the feeding gas comprises hydrogen gas and at least one inert gas. In some embodiments, the feeding gas comprises about 1 vol % to about 10 vol % hydrogen gas, balance being the at least one inert gas. The target liquid products are collected after the reaction is complete, e.g., through solvent extraction in a cold trap. In some embodiments, the solvent extraction uses methanol, ethanol, or acetone, preferably ethanol. In some embodiments, the target liquid products include at least one of benzene, toluene, and/or xylene.

Advantageously, PFRs can be easily tuned for various parameters including, but not limited to, temperature, gas flow, and pressure. Further, catalyst regeneration is fairly simple in a PFR needing only about two hours of air flow.

For catalytic hydrocracking, particularly advantageous catalysts include those containing internal porosity selected according to pore size (e.g., mesoporous and pore sizes typically associated with zeolites). In some embodiments of catalytic hydrocracking, the catalyst may be selected from naturally occurring zeolites, synthetic zeolites and combinations thereof. In some embodiments, the catalyst may be a ZSM-5 zeolite catalyst, as would be understood by those skilled in the art. In some embodiments, such a catalyst can comprise acidic sites. In some embodiments, the zeolite catalysts include, but are not limited to, ferrierite, zeolite Y, zeolite beta, mordenite, MCM-22, ZSM-23, ZSM-57, SUZ-4, EU-1, ZSM-11, (S)AlPO-31, SSZ-23, ZSM-4, ZSM-12, ZSM-20, ZSM-22, ZSM-34, ZSM-35, ZSM-48, and ZSM-50. In some embodiments, product selectivity can be adjusted using zeolite of different pore sizes.

In some embodiments, the catalyst material must be regenerated for further catalytic reactions. In some embodiments, the catalyst material comprises a zeolite, which is washed and dried to separate generated coke and any remaining recondensed heavy olefin products from the zeolite. Thereafter, the dried zeolite catalyst can be regenerated through calcination in air and reused.

In some embodiments, the hydrogen gas is generated in proximity to the upcycling system. For example, in some embodiments, hydrogen gas is generated from the electrolysis of seawater, as well understood by the person skilled in the art. In other words, a portable system can be located on a coastline of, or within, a body of seawater comprising salt and can be a source of marine plastic waste and seawater for the generation of hydrogen gas using electrolysis. In some embodiments, the electrolysis process comprises the use of an electrolyzer, which converts water to hydrogen gas and oxygen gas. In some embodiments, the electrolyzer is one of a polymer electrolyte membrane (PEM) electrolyzer, an alkaline electrolyzer, or a solid oxide electrolyzer. It should be appreciated by the person skilled in the art that the hydrogen gas can be generated using other methods including, but not limited to, steam methane reforming, partial oxidation of heavier hydrocarbons, coal gasification, biogas gasification, and methane pyrolysis. In some embodiments, hydrogen gas is used in the feeding gas. In some embodiments, hydrogen gas is used in the feeding gas and is used to provide energy to other parts of the upcycling system.

It should be appreciated by the person skilled in the art that the upcycling system described herein can be portable, but it is not required. Further, the upcycling system described herein can be located in, e.g., floating in, or near seawater, but it is not required.

There are different ways to collect plastic waste from harbors, water reservoirs (lakes, ponds), rivers (rivers, canals) and coastal areas. In some embodiments, manual collection is used wherein workers maneuver motorized boats and collect the waste with a dip net or other collection means. In some embodiments, mechanized collection is used, wherein vehicles specifically designed to perform clean-up operations, maneuvered by on-board human operators, are able to collect floating wastes by a grid bucket. In some embodiments, robotic collection is used, including wire-guided waste collection modules. Robotic collection can also include aquatic drones that collect floating waste in aquatic areas. Another robot collector accumulates the waste between two hulls. To collect floating plastic waste from the ocean, floating net barriers having a large U shape can be pulled behind two ships.

Accordingly, in a first aspect, a system for upcycling plastic waste to produce aromatic compounds is described, said system comprising:
a plug-flow reactor (PFR) with catalytic hydrocracking functionality, wherein the PFR comprises a reactor tube comprising a catalytic material;
a furnace;
a source of inert gas;
a source of hydrogen gas; and
a cold trap.

In some embodiments of the first aspect, the system for upcycling plastic waste to produce aromatic compounds comprises:
a plug-flow reactor (PFR) with catalytic hydrocracking functionality, wherein the PFR comprises a reactor tube comprising a catalytic material;
a furnace that accommodates the reactor tube;
a source of inert gas that is communicatively connected with an interior of the reactor tube;
a source of hydrogen gas that is communicatively connected with an interior of the reactor tube; and
a cold trap.

In some embodiments of the first aspect, the system for upcycling plastic waste to produce aromatic compounds comprises:
a plug-flow reactor (PFR) with catalytic hydrocracking functionality, wherein the PFR comprises a reactor tube comprising a catalytic material;
a furnace that can be harnessed to heat the reactor tube;
a source of inert gas that is communicatively connected with an interior of the reactor tube;
a source of hydrogen gas that is communicatively connected with an interior of the reactor tube; and
a cold trap.

In some embodiments, the catalytic material can effectively hydrocrack components of the plastic waste into aromatic compounds. In some embodiments, the catalytic material comprises a zeolite. In some embodiments, the furnace can be heated to temperatures in a range from about 500° C. to about 900° C. In some embodiments, the furnace can accommodate the reactor tube. In some embodiments, heat from the furnace is harnessed to heat the reactor tube. In some embodiments, the source of hydrogen gas is an electrolysis system. In some embodiments, the source of hydrogen gas is a seawater electrolysis system. In some embodiments, the cold trap is used for solvent extraction of the aromatic compounds.

In a second aspect, a process of upcycling plastic waste to produce aromatic compounds, said process comprising:
introducing the plastic waste to plug-flow reactor (PFR) reactor tube, wherein the reactor tube comprises a catalytic material;
removing air from the reactor tube using at least one inert gas;
heating the reactor tube and the plastic waste contained therein to a target temperature;
introducing a feeding gas to the reactor tube once the target temperature is achieved; and
collecting the produced aromatic compounds through solvent extraction in a cold trap.

In some embodiments, the catalytic material can effectively hydrocrack components of the plastic waste into aromatic compounds. In some embodiments, the catalytic material comprises a zeolite. In some embodiments, the zeolite is a naturally occurring zeolite, a synthetic zeolite, or a combination thereof. In some embodiments, the plastic waste comprises at least one of low density polyethylene, linear low density polyethylene, high density polyethylene, polypropylene, polyethylene terephthalate, polystyrene, and polyvinyl chloride. In some embodiments, the at least one inert gas is selected from the group consisting of nitrogen, helium, argon, neon, and xenon. In some embodiments, the target temperature is in a range from about 500° C. to about 900° C., preferably about 600° C. to about 700° C. In some embodiments, the furnace can accommodate the reactor tube. In some embodiments, heat from the furnace is harnessed to heat the reactor tube. In some embodiments, the feeding gas comprises hydrogen gas and at least one inert gas. In some embodiments, the feeding gas comprises about 1 vol % to about 10 vol % hydrogen, balance at least one inert gas. In some embodiments, the hydrogen gas is generated using an electrolysis system. In some embodiments, the hydrogen gas is generated using a seawater electrolysis system. In some embodiments, the aromatic compounds are selected from benzene, toluene and xylene. In some embodiments, the process further comprises regenerating the catalytic material for reuse.

### Computer Program Product

The present subject matter described in the first or second aspect may be a system, a method, and/or a computer program product. In some embodiments, the computer program product may include a computer readable storage medium (or media) having computer readable program instructions thereon for causing a processor to carry out aspects of the present subject matter.

In some embodiments, the computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

In some embodiments, computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

In some embodiments, the computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. In some embodiments, the computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In some embodiments, the computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLE

Referring to FIG. 1, the marine plastic waste upcycling was conducted in a plug-flow reactor at atmospheric pressure. A mixture of 200 mg HDPE with and without 50 mg NaCl (to simulate the dried marine plastic waste) was prepared. The reactor tube was loaded with 100 mg of zeolite, and the mixture of HDPE and NaCl was loaded on top the zeolite, separated by quartz wool or some other material that serves as a support for the HDPE on the catalyst. The reactor tube was put in a tubular furnace. An inert gas flow was introduced to the reactor to remove the remaining air and maintain an inert gas atmosphere therein. Once the air was completely removed from the reactor tube, the reactor tube was heated to a target reaction temperature (e.g., 650° C.). As soon as the reaction temperature was reached, a feeding gas was introduced, wherein the feeding gas comprises 5 vol % H₂ with the balance inert gas. The gas products were measured through online and continuous gas chromatography injections. The liquid products (e.g., BTX) were collected after the reaction was complete through solvent (e.g., ethanol) extraction in a cold trap. The recovered zeolite catalyst was washed and dried to separate generated coke and any remaining recondensed heavy olefin products. The dried zeolite catalyst was regenerated through calcination in air.

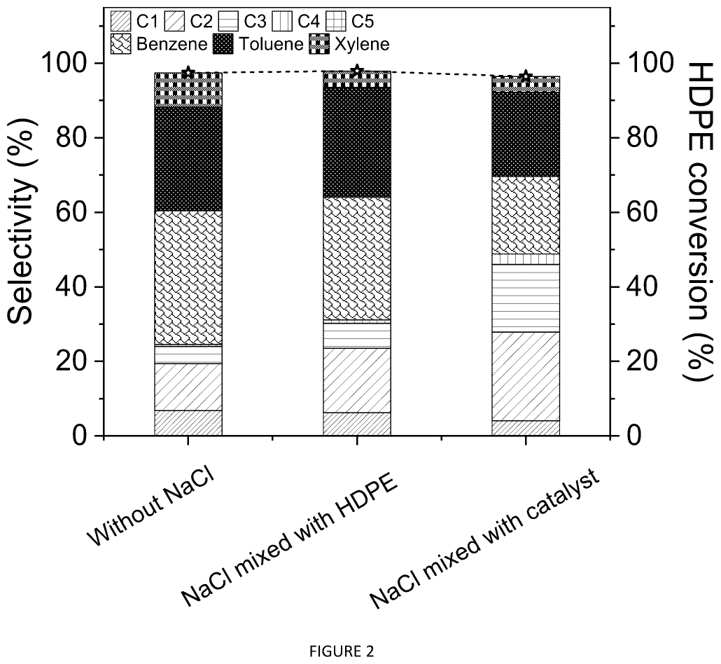

The results are shown in FIG. 2. Without NaCl mixed with HDPE, the BTX yield obtained was 72.86 wt % under optimal reaction conditions. When the HDPE was mixed with NaCl, the BTX yield was 66.79 wt % under the same reaction conditions mentioned above. When the NaCl is mixed with the zeolite catalyst, the BTX yield was 47.60 wt %. In all performance tests, more than 97% of HDPE can be converted into gaseous hydrocarbons and BTX products.

What is claimed is:

1. A system for upcycling plastic waste to produce aromatic compounds, said system comprising:
a plug-flow reactor (PFR) with catalytic hydrocracking functionality, wherein the PFR comprises a reactor tube comprising a catalytic material;
a furnace;
a source of inert gas;
a source of hydrogen gas; and
a cold trap.

2. The system of claim 1, wherein the catalytic material can effectively hydrocrack components of the plastic waste into aromatic compounds.

3. The system of claim 1, wherein the catalytic material comprises a zeolite.

4. The system of claim 1, wherein the furnace can be heated to temperatures in a range from about 500° C. to about 900° C.

5. The system of claim 1, wherein the furnace can accommodate the reactor tube.

6. The system of claim 1, wherein heat from the furnace is harnessed to heat the reactor tube.

7. The system of claim 1, wherein the source of hydrogen gas is an electrolysis system.

8. The system of claim 1, wherein the cold trap is used for solvent extraction of the aromatic compounds.

9. A process of upcycling plastic waste to produce aromatic compounds, said process comprising:
introducing the plastic waste to a plug-flow reactor (PFR) reactor tube, wherein the reactor tube comprises a catalytic material;
removing air from the reactor tube using at least one inert gas;
heating the reactor tube and the plastic waste contained therein to a target temperature;
introducing a feeding gas to the reactor tube once the target temperature is achieved; and
collecting the produced aromatic compounds through solvent extraction in a cold trap.

10. The process of claim 9, wherein the catalytic material can effectively hydrocrack components of the plastic waste into aromatic compounds.

11. The process of claim 9, wherein the catalytic material comprises a zeolite, wherein the zeolite is a naturally occurring zeolite, a synthetic zeolite, or a combination thereof.

12. The process of claim 9, wherein the plastic waste comprises at least one of low density polyethylene, linear low density polyethylene, high density polyethylene, polypropylene, polyethylene terephthalate, polystyrene, and polyvinyl chloride.

13. The process of claim 9, wherein the at least one inert gas is selected from the group consisting of nitrogen, helium, argon, neon, and xenon.

14. The process of claim 9, wherein the target temperature is in a range from about 500° C. to about 900° C.

15. The process of claim 9, wherein the feeding gas comprises hydrogen gas and at least one inert gas.

16. The process of claim 15, wherein the feeding gas comprises about 1 vol % to about 10 vol % hydrogen, balance at least one inert gas.

17. The process of claim 16, wherein the hydrogen gas is generated using an electrolysis system.

18. The process of claim 17, wherein the hydrogen gas is generated using a seawater electrolysis system.

19. The process of claim 9, wherein the aromatic compounds are selected from benzene, toluene and xylene.

20. The process of claim 9, further comprising regenerating the catalytic material for reuse.
